(19) United States
(12) Patent Application Publication
(10) Pub. No.: US 2008/0281111 A1
(43) Pub. Date: Nov. 13, 2008

(54) Process for preparation of mycophenolate mofetil and other esters of mycophenolic acid

(76) Inventors: Sandor Molnar, Debrecen (HU); Csaba Szabo, Debrecen (HU); Tivadar Tamas, Debrecen (HU); Janos Hajko, Debrecen (HU); Claude Singer, Kfar Saba (IL); Beata Kosztya, Debrecen (HU)

Correspondence Address: KENYON & KENYON LLP, ONE BROADWAY, NEW YORK, NY, 10004, US

(21) Appl. No.: 12/218,901

(22) Filed: Jul. 18, 2008

Related U.S. Application Data
(62) Division of application No. 11/115,820, filed on Apr. 26, 2005.

(51) Int. Cl. C07D 307/77

(52) U.S. Cl. 549/468

(57) ABSTRACT

Provided are processes for the preparation of mycophenolate mofetil and other esters of mycophenolic acid.

### CROSS-REFERENCE TO RELATED APPLICATIONS

This application is a divisional of U.S. patent application Ser. No. 11/115,820 and claims the benefits of U.S. Provisional Patent Application Nos. 60/566,056 filed Apr. 27, 2004, 60/572,985 filed May 20, 2004, 60/589,400 filed Jul. 19, 2004, 60/638,478 filed Dec. 23, 2004, 60/639,151 filed Dec. 22, 2004, 60/642,867 filed Jan. 10, 2005, and 60/661,485 filed Mar. 15, 2005, the contents of all of which are incorporated herein by reference.

### BACKGROUND OF THE INVENTION

Mycophenolic acid has the chemical name 6-[4-Hydroxy-6-methoxy-7-methyl-3-oxo-5-phthalanyl]-4-methyl-hex-4-enoic acid, 6-[1,3-Dihydro-4-hydroxy-6-methoxy-7-methyl-3-oxo-isobenzofuran-5-yl]-4-methyl-hex-4-enoic acid, molecular formula of C₁₇H₂₀O₆, molecular weight of 320.35, CAS Registry number of 24280-93-1 and a structure of:

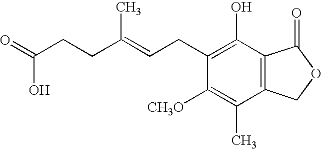

Mycophenolic acid (MPA), isolated by Gosio in 1893, is the first well characterized antibiotic (Bentley 2001). It is produced by several species of Penicillium, including P. brevi-compactum, P. scabrum, P. nagemi, P. roqueforti, P. patris-mei and P. viridicatum (Clutterbuck et al. 1932, Jens and Filtenborg 1983).

MPA, in addition to its antibiotic activity (Abraham 1945), also has antifungal (Gilliver 1946), antiviral (Ando et al. 1968) and antitumor properties (Noto et al. 1969), and has been used clinically in the treatment of psoriasis (Johnson 1972). More recently, it has been recognized as a powerful immunosuppressant (Bentley 2000).

At least one reason for its pharmacological properties is the fact that in several biological systems it interferes with guanine biosynthesis at the level of inosine monophosphate dehydrogenase (IMPD). It has, therefore, a pronounced inhibitory effect on nucleic acid synthesis (Franklin and Cook 1969). The inhibition of IMPD is also the basis of its lymphocyte-specific immunosuppressive effect. Since lymphocytes primarily depend on de novo guanine biosynthesis, the reduction of this pathway results in suppression of T and B lymphocyte proliferation.

MPA was withdrawn due to its high incidence of side effects (primarily infections such as herpes zoster and gastrointestinal side effects such as stomach discomfort). The 2-morpholinoethyl ester derivative, mycophenolate mofetil (CellCept®) does not have these drawbacks, and has a better bioavailability than mycophenolic acid. Mycophenolate mofetil was recently approved (in the United States in 1995 and in Europe in 1996) for prophylaxis of organ rejection in patients receiving allogeneic renal transplants (Shaw and Nowak 1995, Sollinger 1995). After oral administration the ester form rapidly hydrolyzes to free acid. MPA is then converted mainly to an inactive glucuronide metabolite, which is eliminated by urinary excretion (Bentley 2001, Wiwattanawongsa et al. 2001).

Esterification of MPA is known (general methods of esterification are available, e.g. in Synthetic Organic Chemistry by R. B. Wagner and H. D. Zook, Wiley, New York, 1956, see pages 479-532). Mycophenolate mofetil was first disclosed in U.S. Pat. No. 4,753,935. U.S. Pat. No. 5,543,408 discloses the anhydrous crystalline salt, monohydrate salt and amorphous salt forms of mycophenolate mofetil. These forms are characterized by their melting points and/or Differential Scanning Calorimetric results and/or powder X-ray diffraction pattern. Esterification of MPA to prepare mycophenolate mofetil is disclosed in U.S. Pat. No. 5,247,083, WO 00/34503, WO 02/100855, and U.S. Pub. No. 2004/0167130.

When converting MPA to mycophenolate mofetil according to the process disclosed in WO 00/34503, it was observed that a large part of the MPA is left unconverted, and impurities are formed.

There is a need in the art for additional processes for preparation of mycophenolate mofetil and other esters of MPA.

### SUMMARY OF THE INVENTION

In one aspect the present invention provides a process for preparing an ester of mycophenolic acid comprising:

reacting a mycophenolic acid of formula:

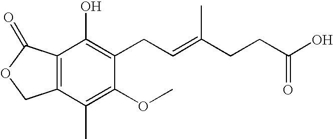

with a C₁ to C₄ alcohol or 4-(2-hydroxyethyl)morpholine in the presence of a catalyst, to obtain an ester of mycophenolic acid of formula:

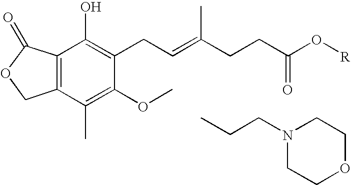

wherein R is C₁ to C₄ alkyl or a group.
In one embodiment, the reaction is carried out in the absence of a solvent. In one embodiment, the alcohol is 4-(2-hydroxyethyl)morpholine. In one embodiment, the alcohol is a C₁ to C₄ alkanol. In one embodiment, the alcohol is methanol, ethanol, isopropanol, or isobutanol. alcohol is present in an amount of about 1 to about 6 molar equivalents of the mycophenolic acid. In one embodiment, the alcohol is present in an amount of about 3 to about 6 molar equivalents of the mycophenolic acid.

In another aspect, the present invention provides a process for preparing mycophenolate mofetil, comprising the step of reacting mycophenolate C₁ to C₄ alkyl ester with 4-(2-hydroxyethyl)morpholine, in the presence of a catalyst and without a solvent.

The catalyst for the processes of the present invention may be selected from the group consisting of: tin(II) chloride, iron(II) chloride, zinc sulfate, camphorsulfonic acid, and potassium dihydrogenphosphate. More preferably the catalyst is selected from the group consisting of tin(II) chloride, iron(II) chloride and zinc sulfate. Most preferably the catalyst is tin(II) chloride. In one embodiment the catalyst is present in an amount of about 0.005 to about 0.2 molar equivalents of the mycophenolic acid. In one embodiment, the catalyst is present in an amount of about 0.15 molar equivalents of the mycophenolic acid. In one embodiment, the reaction is carried out under inert atmosphere. In one embodiment, the reaction is carried out at a temperature of about room temperature to about reflux temperature. In one embodiment, the reaction is carried out at a temperature of about 30° C. to about 200° C. In one embodiment, the reaction is carried out at a temperature of about 140° C. to about 180° C.

In another aspect the present invention provides a process for preparing mycophenolate mofetil comprising:
a) reacting 4-(2-hydroxyethyl)morpholine with mycophenolic acid in the presence of a catalyst and absence of a solvent to obtain mycophenolate mofetil;
b) combining the mycophenolate mofetil with a water-immiscible solvent to form an aqueous phase and a water-immiscible phase;
c) extracting the water-immiscible phase with an aqueous alkaline solution to remove mycophenolic acid;
d) extracting the water-immiscible phase with water to remove 2-(4-morpholinyl)ethyl (E)-6-(1,3-dihydro-4-[2-(4-morpholinyl)ethoxy]-6-methoxy-7-methyl-3-oxo-isobenzofuran-5-yl)-4-methyl-hex-4-enoate (Compound 1); and
e) crystallizing the mycophenolate mofetil.
   Also provided are pharmaceutical compositions of the mofetil obtained in this process and its use in a method of suppressing the immune system of a mammal in need thereof.

### DETAILED DESCRIPTION OF THE INVENTION

As used herein, the term “mixture” includes both heterogeneous and homogenous mixtures, such as, for example, a solution, suspension, or slurry. A heterogeneous mixture may be formed, for example, during extraction, where mycophenolic acid is dissolved in a solvent by basification.

As used herein, the term “alkaline” or “basic” refers to a pH of greater than 7.

As used herein, the term “acidic” refers to a pH of less than 7.

The invention encompasses processes for preparing mycophenolate mofetil and other esters of MPA in a catalytic reaction. The catalyst used may be a particular Lewis acid catalyst.

Certain Lewis acid catalysts are able to change the direction of the process in such a way that an advanced conversion of mycophenolic acid can be achieved while maintaining at the same time the impurity 2-(4-morpholinyl)ethyl (E)-6-(1,3-dihydro-4-[2-(4-morpholinyl)ethoxy]-6-methoxy-7-methyl-3-oxo-isobenzofuran-5-yl)-4-methyl-hex-4-enoate,

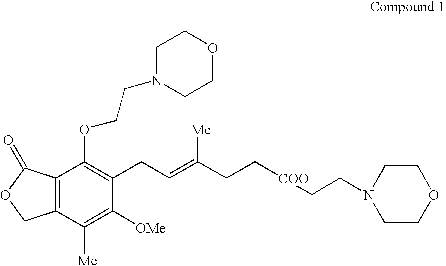

(designated Compound 1) at a level which facilitates its subsequent removal from the drug.

In one embodiment, the catalytic process for preparing esters of mycophenolic acid, preferably mycophenolate mofetil, is performed with or without a solvent, under an inert atmosphere. This process comprises:

reacting a mycophenolic acid of formula (I):

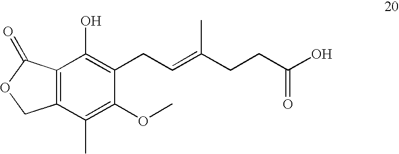

with a C₁ to C₄ alcohol or 4-(2-hydroxyethyl)morpholine in the presence of a catalyst, to obtain an ester of mycophenolic acid of formula (II):

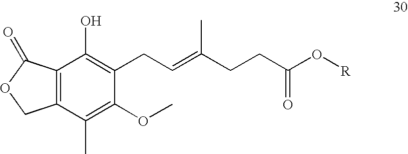

wherein R is C₁ to C₄ alkyl or a

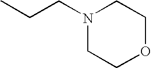

group.

As used herein, the term “inert atmosphere” refers to unreactive atmospheres, which includes, for example, nitrogen or argon atmosphere

Preferably, the reaction is carried out neat, i.e. in the absence of a solvent.

A preferable C₁ to C₄ alcohol is methanol, ethanol, isopropanol or isobutanol. The C₁ to C₄ alcohol or 4-(2-hydroxyethyl)morpholine used should be in an amount sufficient to produce mycophenolate esters of the invention. Preferably, an amount of about 1 to about 6 molar equivalents of the mycophenolic acid, and more preferably about 3 to about 6 molar equivalents.

In another embodiment, mycophenolate mofetil is prepared by reaction of a C₁ to C₄ alkyl ester of mycophenolic acid with 4-(2-hydroxyethyl)morpholine without a solvent in the presence of a catalyst.

The 4-(2-hydroxyethyl)morpholine should be in an amount sufficient to produce the mycophenolate mofetil. Preferably, the 4-(2-hydroxyethyl)morpholine is in an amount of about 1 to about 6 molar equivalents of the mycophenolic acid, and more preferably about 3 to about 6 molar equivalents.

Acid catalysts favor an esterification reaction. However, not all acid catalysts have the same effect on the reaction selectivity of mycophenolic acid with morpholine ethanol. Whereas catalysts such as tin(II) chloride, iron(III) chloride, or zinc sulfate; or organic acids such as camphorsulfonic acid; or other inorganic salts such as potassium dihydrogenphosphate; can be used to promote the esterification reaction, not all catalysts favor the conversion and increase the selectivity of the esterification reaction towards the desired compound.

Catalysts that do favor conversion and increase selectivity include certain Lewis acid catalysts such as, for example, tin(II) chloride, iron(III) chloride, or zinc sulfate. A most preferable catalyst is tin(II) chloride. The catalyst should be in an amount sufficient to increase the reaction speed and selectivity. Preferably, the catalyst is present in an amount of about 0.005 to about 0.2 molar equivalents of the mycophenolic acid, and more preferably about 0.15 molar equivalents.

The reaction should be at a suitable temperature to move the reaction forward. Generally, the reaction temperature may be from room temperature to about reflux temperature. Preferably, the reaction temperature is about 30° C. to about 200° C., and more preferably about 140° C. to about 180° C. Generally, reaction time depends on factors such as the reagents, temperature, or the amount of reagents. Preferably, the reaction time is about 1.5 to about 10 hours, and more preferably about 4 to about 9 hours.

The reaction mixture may undergo various treatments, such as, for example, extraction, washing, decolorization, or filtration, to obtain a crude product. The crude product is then crystallized at least once from a suitable solvent or solvent mixture.

The extraction process facilitates removal of significant impurities. Removal of impurities refers to reducing the levels of impurities as defined by European Pharmacopoeia.

Unreacted mycophenolic acid may be removed, for example, by alkaline extraction. The alkaline extraction may be carried out, for example, by admixing the mycophenolate ester with a water-immiscible solvent and extracting the ester with an alkaline aqueous solution.

Any water-immiscible solvent suitable for extracting the mycophenolate ester may be used. Examples of suitable solvents include, but are not limited to, at least one of ethyl acetate, isobutyl acetate, methyl ethyl ketone, or toluene.

An alkaline aqueous solution may be prepared, for example, from sodium bicarbonate, sodium carbonate, or sodium hydroxide. The alkaline extraction is carried out at a pH of about 7 to about 12, and preferably at about 8 to about 10.

The impurity designated Compound 1, may be removed by acidic extraction, as described in commonly-owned U.S. application Ser. No. 11/______ [K&K ref: 2664/58504 filed 26 Apr. 2005. The U.S. Ser. No. will be completed when available]. This acidic extraction method comprises: admixing the mycophenolate ester with a water-immiscible solvent; washing the mycophenolate mofetil admixture with an aqueous acidic solution to obtain a two-phase system; separating the organic phase containing mycophenolate mofetil from the aqueous acidic phase; adding an aqueous basic solution to the aqueous acidic phase; and recovering Compound 1.

After extraction, a residue is obtained by concentration, and is crystallized from at least one solvent. The residue may be obtained by evaporation at atmospheric or reduced pressure, preferably at below 1 atm, and more preferably at below about 100 mm Hg.

An anti-solvent such as isopropanol may be added to the mixture of the mycophenolate ester in the water-immiscible solvent obtained after extraction for optimum crystallization. The anti-solvent may also be added after concentration into the residue.

Water may also be added to the reaction mixture obtained from the extraction, and the mixture is seeded.

Crystallization helps remove other known significant impurities such as, for example, impurity A as defined by European Pharmacopoeia, or the lactone or Z-isomer of the mycophenolic ester.

Any solvent suitable for crystallization may be used. Examples of suitable solvents include, but are not limited to, ketones (such as acetone or methyl ethyl ketone), alcohols (such as methanol, ethanol, n-propanol, or isopropanol), esters (such as ethyl acetate or isobutyl acetate), ethers (such as diisopropyl ether or tert-butyl methyl ether), or other solvents such as acetonitrile or toluene. The above solvents may also be mixed with ethers, alcohols, or alkanes (such as n-heptane, n-hexane or cyclohexane). Preferred solvents include acetone/isopropanol, isobutyl acetate, isobutyl acetate/isopropanol, isobutyl acetate/acetone/isopropanol, acetonitrile/isopropanol, or toluene/isopropanol.

Crystallization is carried out a suitable temperature to dissolve the crude product. The solution may be heated, preferably at about 30° C. to about 60° C., and more preferably at about 40° C. to about 45° C. The solution may then be cooled, preferably at about −10° C. to about 10° C., and more preferably at about −5° C. to about 0° C. The cooling time may vary depending on the crystallization conditions. Preferably, the solution is cooled for about 2 to about 10 hours, and more preferably about 6 hours. The solution is then allowed to crystallize, preferably for about 2 to about 20 hours, and more preferably for about 10 to about 12 hours. The recovered solid may be dried at atmospheric or reduced pressure, preferably at about 40° C. to about 80° C., and more preferably at about 60° C.

Mycophenolic acid used to prepare the ester in the present invention may be prepared by any methods known in the art. See, e.g., WO 01/21607, WO 01/64931 and GB 1158387. MPA may be also prepared by the processes disclosed in commonly-owned U.S. application Ser. No. 11/______ [K&K ref: 2664/60903, which is filed on Apr. 26, 2005. The U.S. Ser. No. will be completed when available], which process comprises:
a) admixing a concentrated alkaline mixture containing mycophenolic acid with a first water-immiscible solvent to form an aqueous phase and a first water-immiscible phase;
b) separating the aqueous phase;
c) admixing the aqueous phase with a second water-immiscible solvent at a pH of less than about 7 to form an aqueous phase and a second water-immiscible phase;
d) separating the second water-immiscible phase;
e) concentrating the second water-immiscible phase; and
f) crystallizing mycophenolic acid.

The second water-immiscible phase in step e) is preferably concentrated by membrane filtration.

The concentrated alkaline mixture in step a) may be prepared from a fermentation broth by various methods. Preferably, it is obtained by the method comprising: basifying a fermentation broth containing mycophenolic acid, and removing the mycelia to obtain a basic mixture; acidifying the basic mixture to obtain an acidic mixture; and filtering and basifying the acidic mixture, to obtain the concentrated alkaline mixture.

In one embodiment, the mycophenolate mofetil prepared by the processes of the present invention has about 0.01 to about 0.1% of Compound 1 as measured by HPLC area percentage. The processes provided in the present invention further comprise the step of formulating the ester of mycophenolic acid with one or more pharmaceutical acceptable excipients.

### Pharmaceutical Compositions

Pharmaceutical formulations of the invention contain mycophenolic acid ester, and preferably the mofetil ester. Also included are pharmaceutically acceptable salts of the mofetil ester such as, for example, acetic, benzoic, fumaric, maleic, citric, tartaric, gentisic, methane-sulfonic, ethanesulfonic, benzenesulfonic and laurylsulfonic, taurocholat, hydrobromide, or hydrochloride salts. The pharmaceutical composition may contain a single polymorphic form, or a mixture of various crystalline forms, with or without amorphous form.

In addition to the active ingredient(s), the pharmaceutical composition may contain one or more excipients or adjuvants. Selection of excipients and the amounts may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, nateglinide and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid, bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The invention also encompasses a method of suppressing the immune system of a mammal by administering a therapeutically effective amount of the pharmaceutical composition to a mammal in need thereof.

### EXPERIMENTAL

### Analytical HPLC Method

An assay is a determination of the purity or presence of a quantity of a substance, as described by the European Pharmacopoeia (“EP”). EUROPEAN PHARMACOPOEIA, 4^{th} ed., Council of Europe, Strasbourg, 2001. The assay is performed by high pressure liquid chromatography (“HPLC”). HPLC methods are carried out according to Pharmaeuropa.

HPLC analysis was conducted using a Discovery ciano or Zorbax C₈ column The eluent was a water-acetonitrile mixture containing phosphoric acid and the potassium salt of phosphoric acid. The triethylamine salt of phosphoric acid may be used in place of the potassium salt of phosphoric acid. The pH of the eluent was 3.0-5.9. The eluent flow was approximately 1.5 ml/min. The temperature for elution was 20-45° C.

### Preparation of Mycophenolate Mofetil

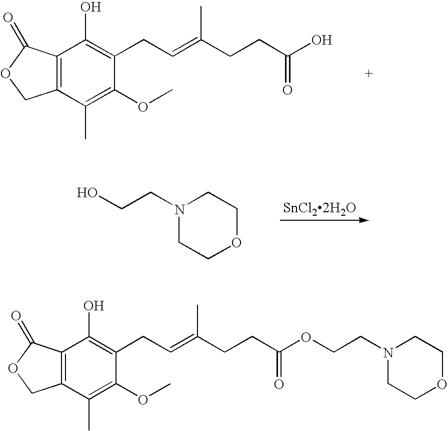

### Example 1

A mixture of mycophenolic acid (192 g, 0.6 mol) and 4-(2-hydroxyethyl)morpholine (440 ml, 6 molar equivalents) was stirred at 150-155° C. for 4 hours in the presence of tin(II) chloride dihydrate (20.4 g, 0.15 molar equivalents) under nitrogen atmosphere. After the completion of the reaction, the reaction mixture was allowed to cool to room temperature. The obtained dark liquid was poured into isobutyl acetate (4.0 l). The solution was extracted with 2% of aqueous sodium bicarbonate solution (1.2 l, then 2×0.4 l). After the first addition of sodium bicarbonate solution, the formed two-phase system was treated with charcoal (40 g) and filtrated (an emulsion was filtered off). The solution was extracted with water (1 liter). After phase separation the organic phase was washed with water (1 liter) and evaporated to dryness at 40-50° C. under vacuum. To the solid material acetone (400 ml) and isopropanol (3.8 l) were added and the mixture was warmed to 40-45° C. The material was dissolved. The solution was cooled to −5° C. over 6 hours and stirred at this temperature for 10-12 hours. After filtration, the crystals were washed with a 2:19 acetone/isopropanol mixture (420 ml). The crude compound was dried under vacuum at 60° C. The yield was 169-195 g (65-75%).

MPA level: 0.1 area %. Assay: 99.85%.

### Example 2

A mixture of mycophenolic acid (9.60 g, 30 mmol), 4-(2-hydroxyethyl)morpholine (14.7 ml, 4 molar equivalents) and (+)-camphorsulfonic acid (0.21 g, 0.9 mmol, 3 mol %) was stirred at 150-155° C. for 8 hours. After cooling to room temperature, water (200 ml) was added to the reaction mixture, and the mixture was seeded and stirred for 2 hours. The solid material was filtered off, washed with water (100 ml) and dried at room temperature. The product was 10.93 g (84% yield).

MPA level: 2.4 area %.

### Example 3

A mixture of mycophenolic acid (64.07 g, 0.2 mol), 4-(2-hydroxyethyl)morpholine (98 ml, 4 molar equivalents) and potassium dihydrogenphosphate (0.82 g, 3 mol %) was stirred under nitrogen atmosphere at 165° C. for 3 hours. The cooled mixture was dissolved in toluene (700 ml) at room temperature, and the solution was washed with 5% aqueous sodium bicarbonate solution (2×700 ml). The organic phase after drying on sodium sulfate was decolorized with charcoal (30 g). To the stirred solution, n-heptane (1000 ml) was added and the mixture was warmed to 60° C. The solution was cooled to −10° C., and after 1 hour the crystals were filtered off and dried at room temperature. The crude product was 54.0 g (62% yield).

MPA level: 0.06 area %. Assay: 95.6%

### Example 4

A mixture of mycophenolic acid (9.60 g, 30 mmol), 4-(2-hydroxyethyl)morpholine (14.7 ml, 4 molar equivalents) and tin(II) chloride dihydrate (0.20 g, 3 mol %) was stirred under nitrogen atmosphere at 180° C. for 90 minutes. The cooled mixture was diluted in toluene (100 ml) at room temperature, and the solution was washed with saturated sodium bicarbonate solution (100 ml). The aqueous washing liquor was re-extracted with toluene (25 ml). The organic phases were combined, washed with saturated sodium bicarbonate solution (2×100 ml), dried on sodium sulfate, decolorized with charcoal (1 g) and evaporated to the 1/3 volume (ca. 44 g). Isopropanol (150 ml) was added to the solution and the mixture was kept in the fidge overnight. The solid was filtered off, washed with heptane (15 ml) and dried at room temperature. The crude product was 8.54 g (65% yield).

MPA level: 0.06 area %. Assay: 97.1%

### Example 5

A mixture of mycophenolic acid (9.60 g, 30 mmol), 4-(2-hydroxyethyl)morpholine (22.0 ml, 6 molar equivalents) and zinc sulfate heptahydrate (0.04 g, 0.5 mol %) was stirred under nitrogen atmosphere at 160-165° C. for 4 hours. The cooled mixture was dissolved in toluene (100 ml) at room temperature, and the solution was washed saturated sodium bicarbonate solution (100 ml). The aqueous washing liquor was re-extracted with toluene (25 ml). The organic phases were combined, washed with saturated sodium bicarbonate solution (2×100 ml), dried on sodium sulfate, decolorized with charcoal (1 g) and evaporated to the 1/3 volume (ca. 38 g). To the solution isopropanol (150 ml) was added and the mixture was kept in the fridge overnight. The solid was filtered off, washed with heptane (15 ml) and dried at room temperature. The crude product was 8.80 g (68% yield).

MPA level: 0.03 area %. Assay: 96.0%

### Crystallization of Mycophenolate Mofetil

### Example 6

The crude compound (172 g) was dissolved in acetone (344 ml) and isopropanol (3.27 l) at 40-45° C. The warmed solution was treated with charcoal (17.2 g, 10%). After filtration the solution was cooled to −5° C. over 6 hours and stirred at this temperature for 10-12 hours. The precipitated crystals were filtered off and washed with 2:19 acetone/isopropanol mixture (361 ml). The crystallized compound was dried under vacuum at 60° C. The solid was 155-164 g (85-90%).

MPA level: 0.01 area %. Assay: 99.39%

### Example 7

The crude compound (5 g) was dissolved in isobutyl acetate (10 ml) and isopropanol (90 ml) at 40-45° C. The warmed solution was treated with charcoal (0.5 g, 10%). After filtration the solution was cooled to −5° C. over 6 hours and stirred at this temperature for 10-12 hours. The precipitated crystals were filtered off and washed with 1:9 isobutyl acetate/isopropanol mixture (10 ml). The crystallized compound was dried under vacuum at 60° C. The compound was 4.1-4.3 g (82-86%).

MPA level: 0.08 area %. Assay: 98.9%

### Example 8

The crude compound (5 g) was dissolved in isobutyl acetate (100 ml) at 40-45° C. The warmed solution was treated with charcoal (0.5 g, 10%). After filtration the solution was cooled to −5° C. during 6 hours and stirred at this temperature for 10-12 hours. The precipitated crystals were filtered off and washed with isobutyl acetate (10 ml). The crystallized compound was dried in vacuum at 60° C. The solid was 3.55-3.80 g (71-76%). MPA level: 0.11 area %. Assay: 99.7%.

### Example 9

The crude compound (5 g) was dissolved in isobutyl acetate (10 ml), acetone (9 ml) and isopropanol (86 ml) at 40-45° C. The warmed solution was treated with charcoal (0.5 g, 10%). After filtration the solution was cooled to −5° C. over 6 hours and stirred at this temperature for 10-12 hours. The precipitated crystals were filtered off and washed with isobutyl acetate/acetone/isopropanol mixture (10 ml). The crystallized compound was dried under vacuum at 60° C. The solid was 4.05-4.3 g (81-86%).

MPA level: 0.13 area %. Assay: 100.5%.

### Example 10

### Comparative Example in the Absence of a Catalyst

A mixture of mycophenolic acid (9.6 g, 30 mmol), 4-(2-hydroxyethyl)morpholine (147 ml, 4 molar equivalents) was stirred at 160° C. for 5 hours. After cooling to room temperature, water (200 ml) was added, followed by seeding. The solid was filtered off and washed with water. The dry product was 10.9 g (84%).

Crude product: MPA 3 area %, Compound 1: 0.2 area %.

### Example 11

### Comparative Example for the Use of Conventional Catalyst

A mixture of mycophenolic acid (96.1 g, 0.3 mol), 4-(2-hydroxyethyl)morpholine (147 ml, 4 molar equivalents), toluene-4-sulfonic acid monohydrate (1.7 g, 20 mol %) was stirred at 160° C. for 6 hours. The cooled mixture was poured into a stirred mixture of sodium hydrogen carbonate (100 g), Celite 545 (100 g), seeding crystals in 2 L of water. Stirring was continued for 4 hours at room temperature, then the solid was filtered off, washed with water (0.5 L), and dried at room temperature. The crude product was 209.07 g (84%). 2664/610051

Crude product: MMF 91 area %, MPA 2 area %, Compound 1: 0.4 area %.

### Example 12

### Preparation of Methyl Ester of Mycophenolic Acid (Methyl Mycophenolate)

A mixture of mycophenolic acid (9.6 g, 30 mmol) and tin(II) chloride dihydrate (1.0 g, 0.15 molar equivalents) in methanol (40 ml) was stirred at reflux temperature for 7 hours, then evaporated to dryness. The residue was dissolved in isobutyl acetate (300 ml), saturated sodium bicarbonate solution (100 ml) and charcoal (0.5 g) were added. The mixture was filtered and the phases were separated. The organic phase was dried on sodium sulfate, then evaporated to dryness. The weight of the obtained white solid—methyl mycophenolate—was 9.38 g (94% yield). Crude product: MPA 0.45 area %, MPA-Me 98.9 area %.

### Example 13

### Preparation of Mycophenolate Mofetil by Ester Exchange Reaction

A mixture of methyl mycophenolate (10.02 g, 30 mmol), 4-(2-hydroxyethyl)morpholine (22 ml, 6 molar equivalents) and tin(II) chloride dihydrate (1.0 g, 0.15 molar equivalents) were stirred under nitrogen atmosphere at 150° C. for 6 hours, then at 160° C. for 3 hours. The cooled mixture was dissolved in toluene (250 ml) at room temperature, then saturated sodium bicarbonate solution (100 ml) and charcoal (2 g) were added, the mixture was filtered and the phases were separated. After drying on sodium sulfate the organic phase was evaporated to dryness. The solid residue—crude mycophenolate mofetil—was 10.0 g (77% yield).

Crude product: MPA 0.1 area %, MMF 93.8 area %, Compound 1: 1.1 area %.

### Example 14

### Preparation of Mycophenolic Acid Having a Purity of 99.8%

Concentrated mycophenolic acid suspension of 140 kg (produced from 620 kg fermented broth) was pH adjusted with 800 ml conc. ammonium hydroxide solution. The achieved pH was 8.3-8.5. The alkaline solution was purified with 80 liters ethylacetate. The ethylacetate was mixed with the alkaline solution, stirred for 30 minutes, and the phases were separated.

To the obtained (147 kg) aqueous phase, 80 liters of ethylacetate was added. The pH was adjusted to 5.8 with sulfuric acid. Stirring was applied for 30 minutes, and the phases were separated.

To the obtained (150 kg) aqueous phase, 40 liters of ethylacetate was added. The pH was adjusted to 5.9. Stirring was applied for 30 minutes, and the phases were separated.

Obtained ethylacetate phases of the two acidic extractions were combined and concentrated to approx. 200 g/l concentration at max. 70° C. under reduced pressure.

Concentrated ethylacetate solution was heated to 60-65° C. and cooled to −10° C. at a cooling rate of approx. 3° C./hours, and crystallized for 18 hours at −10° C. Then crystals were filtered, coverwashed with cooled ethylacetate, and dried at max. 70° C. under reduced pressure.

Mass of crystals: 1250 g. Assay: 99.0%.

The crystals were recrystallized from ethylacetate after charcoal treatment. Assay: 99.6%. HPLC purity: 99.8 area %. Any impurity is less than 0.1 area %.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated by reference in their entirety.

1-7. (canceled)

8. A process for preparing mycophenolate mofetil, comprising the step of: reacting mycophenolate C₁ to C₄ alkyl ester with 4-(2-hydroxyethyl) morpholine, in the presence of a catalyst and without a solvent.

9-29. (canceled)
